(19) United States
(12) Patent Application Publication
(10) Pub. No.: US 2013/0289047 A1
(43) Pub. Date: Oct. 31, 2013

(54) HETEROARYLTHIO DERIVATIVES AND ANALOGUES

(71) Applicants: David Helton, Dana Point, CA (US); David Fick, Coto de Caza, CA (US)

(72) Inventors: David Helton, Dana Point, CA (US); David Fick, Coto de Caza, CA (US)

(73) Assignee: EPIOMED THERAPEUTICS, INC., Irvine, CA (US)

(21) Appl. No.: 13/879,142

(22) PCT Filed: Oct. 14, 2011
(86) PCT No.: PCT/US11/56479

§ 371 Date: Jul. 9, 2013

Related U.S. Application Data
(60) Provisional application No. 61/393,349, filed on Oct. 14, 2010.

(51) Int. Cl. C07D 233/84; C07D 257/04; C07D 491/04; C07D 413/04

(52) U.S. Cl. CPC C07D 233/84; C07D 413/04; C07D 257/04; C07D 491/04
USPC 514/253.04; 544/370; 514/254.05; 544/364; 514/253.1; 544/366; 544/362

(57) ABSTRACT

Heteroarylthio compounds covalently linked to an arylpiperazine moiety for the treatment of neurological conditions.

### CROSS-REFERENCE TO RELATED APPLICATIONS

This patent application claims the benefit of priority from U.S. Patent Application No. 61/393,349, filed on Oct. 14, 2010 and entitled HETEROARYLTHIO DERIVATIVES AND ANALOGUES. The disclosure of this application is hereby incorporated by reference herein in its entirety.

### BACKGROUND

The serotonin receptors, also known as 5-hydroxytryptamine receptors or 5-HT receptors, are a group of G protein-coupled receptors (GPCRs) and ligand-gated ion channels (LGICs) found in the central and peripheral nervous systems that bind the endogenous neurotransmitter serotonin (5-hydroxytryptamine, 5-HT). They mediate both excitatory and inhibitory neurotransmission. The serotonin receptors are activated by the neurotransmitter serotonin, which acts as their natural ligand.

The 5-HT1 subclass of 5-HT receptors include inhibitory Gi/Go-protein coupled receptors, with binding to this receptor resulting in decreasing cellular levels of cAMP. The 5-HT1A receptor mediates inhibitory neurotransmission, and has been associated with physiological processes and conditions including addiction, aggression, anxiety, appetite, blood pressure, emesis, heart rate, memory, mood, nausea, respiration, sleep, thermoregulation, and vasoconstriction. Other 5-HT receptors, such as the 5-HT7 receptor, mediate excitatory neurotransmission and binding to such receptors can stimulate the production of the intracellular signaling molecule cAMP.

### SUMMARY

The present compounds bind to serotonin receptors, in particular 5HT1A, and have been found to be useful in the treatment of neurological conditions. These compounds have the general schematic structure, {A}-L-{B}, where A is a heteroaryl ring substituted by a thio group, L is a hydrocarbyl chain attached to A through the thio group, and B is an arylpiperazine or arylpiperazine derivative. The present heteroarylthio compounds have the following formula:

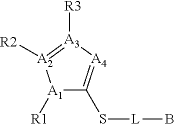

where:

(a) A1 is N, O, or S;

(b) R1 is present when A1 is N and is H, alkyl, aryl, heteroaryl, aralkyl, or heteroaralkyl, each of which may be optionally substituted;

(c) when A1 is O or S, A2 is C and A3 and A4 are C or N;

(d) when A1 is N, A2, A3 and A4 are C or N;

(e) R2 and R3 are present when A2 and A3 are C respectively;

(f) R2 and R3 are H, alkly, amino, carboxamido, sulphonamido, alkylthio, aryl, or heteroaryl, each of which may be optionally substituted; and

(g) R2 and R3 can be taken together to form a six-member aromatic ring which may be optionally substituted;

(h) L is (CH₂)ₘ, wherein m is an integer from 1 to 6; and

(i) B has the following formula:

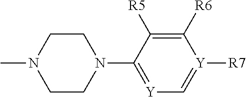

where:
(1) Y is C or N;
(2) R5 is hydrogen, alkyl, hydroxy, halo, alkoxy, cyano, methylthio; nitro, trifluoromethyl, or cycloalkyl;
(3) R6 is hydrogen, alkyl, hydroxy, halo, alkoxy, trifluoromethyl, nitro, amino, aminocarbonyl, or aminosulfonyl;
(4) R5 and R6 can be taken together to form a 5 or 6 member aromatic or non-aromatic ring, which can contain from 0 to 3 heteroatoms selected from the group of N, O, or S, of which the N may be further substituted if it is secondary; and
(5) R7 is hydrogen, alkyl, halo, alkoxy, or trifluoromethyl, or is absent if Y is N.

The linker, L, can be substituted with alkyl groups, and is preferably a chain of 2, 3, or 4 carbons.

In one embodiment, the heteroarylthio compound can have the following formula:

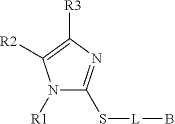

where:
(a) R₁ is H, alkyl, aralkyl, heteroaralkyl, aryl or heteroaryl, each of alkyl, aralkyl, heteroaralkyl, aryl or heteroaryl which may be optionally substituted, and
(b) R₂ and R₃ are each independently selected from H, alkyl, aralkyl, aryl, heteroaryl or R₂ and R₃ may be taken together to form a six-member aromatic ring, each of which may be optionally substituted.

Alternatively, the heteroarylthio compound can have the following formula:

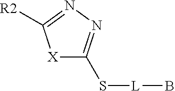

where:
(a) R₂ is alkly, aralkyl, heteroaralkyl, amino, carboxamido, sulphonamido, alkylthio, aryl, or heteroaryl, each of alkly, aralkyl, heteroaralkyl, amino, carboxamido, sulphonamido, alkylthio, aryl, or heteroaryl which may be optionally substituted; and
(b) B is O or S.

In another embodiment, the heteroarylthio compound can have the following formula:

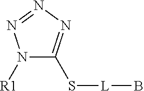

where R₁ is H, alkly, aralkyl, heteroaralkyl, aryl or heteroaryl, each of alkly, aralkyl, heteroaralkyl, aryl or heteroaryl which may be optionally substituted.

In a further embodiment, the heteroarylthio compound can have the following formula:

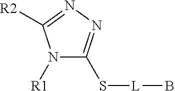

where:
(a) R₁ is H, alkly, aralkyl, heteroaralkyl, aryl or heteroaryl, each of the alkly, aralkyl, heteroaralkyl, aryl or heteroaryl which may be optionally substituted; and
(b) R₂ is H, alkyl, aralkyl, heteroaralkyl, amino, carboxamido, sulphonamido, aryl or heteroaryl, each of alkyl, aralkyl, heteroaralkyl, amino, carboxamido, sulphonamido, aryl or heteroaryl which may be optionally substituted.

In yet another embodiment, the heteroarylthio compound can have the following formula:

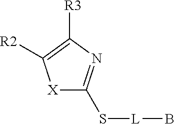

where:
(a) R₂ and R₃ are independently H, alkyl, aralkyl, heteroaralkyl, aryl, heteroaryl or R₂ and R₃ are taken together to form a six-member aromatic ring, each of which may be optionally substituted; and
(b) X is O or S.

In the present compounds, the B moiety is an arylpiperazine moiety, such as one of the following:

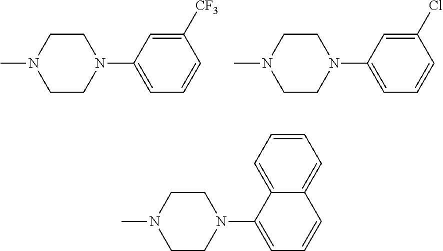

In one embodiment, the B moiety has the following formula:

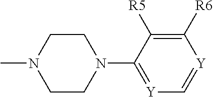

where:
(a) the 6-member heterocyclic ring can be 2-pyridyl, 4-pyridyl, or 4-pyrimidyl; and
(b) R5 and R6 can be taken together to form a 5 or 6 member aromatic or non-aromatic ring, which can contain from 0 to 3 heteroatoms selected from the group of N, O, or S, of which the N may be further substituted if it is secondary

In another embodiment, the B moiety has the following formula:

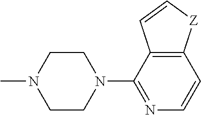

where Z is O or S.

In preferred embodiments, the heteroarylthio compound can be one of the following compounds:
1-{2-[(1-methyl-1H-imidazol-2-yl)thio]ethyl}-4-[3-(trifluoromethyl) phenyl]piperazine;
1-[2-(5-pyridin-4-yl-[1,3,4]oxadiazol-2-ylsulfanyl)ethyl]-4-(3-trifluoromethylphenyl)piperazine;
1-{3-[(1-methyl-1H-imidazol-2-yl)thio]propyl}-4-[3-chlorophenyl]piperazine;
1-[3-(1-phenyl-1H-tetrazol-5-ylsulfanyl)propyl]-4-(3-chlorophenyl)piperazine;
1-[2-(5-pyridin-4-yl-[1,3,4]oxadiazol-2-ylsulfanyl)ethyl]-4-(3-trifluoromethylphenyl)piperazine;
4-{4-[3-(1-methyl-1H-imidazol-2-ylsulfanyl)propyl]piperazin-1-yl}furo[3,2-c]pyridine;
4-{4-[3-(1-phenyl-1H-tetrazol-5-ylsulfanyl)propyl]piperazin-1-yl}furo[3,2-c]pyridine; or
4-{4-[3-(5-pyridin-4-yl-[1,3,4]oxadiazol-2-sulfanyl)propyl]piperazin-1-yl}furo[3,2-c]pyridine.

In another aspect, the present heteroarylthio compounds can be used to treat a neurological condition. Preferably the compounds in this case are admixed with one or more pharmaceutically acceptable excipients in order to produce a pharmaceuical composition. Such a composition can be administered to a subject in need thereof in order to treat the subject.

### FIGURES

FIG. 1 is a graph showing the results of a Condition Avoidance Responding test using one of the present compounds.

FIG. 2 is a graph showing the results of another Condition Avoidance Responding test using another one of the present compounds.

### DETAILED DESCRIPTION

### I. Definitions

As used herein, the following terms and variations thereof have the meanings given below, unless a different meaning is clearly intended by the context in which such term is used.

“About” when used in reference to a numerical value means plus or minus ten percent of the indicated amount. For example and not by way of limitation, “about 10” means between 9 and 11, and “about 10%” means between 9% and 11%.

“Agonist” means a material (e.g., molecule, compound, or other material) that activates an intracellular response when it binds to a receptor.

“Partial agonist” means a material (e.g., molecule, compound, or other material) that activates an intracellular response when it binds to a receptor to a lesser degree/extent than do agonists, or enhances GTP binding to membranes to a lesser degree/extent than do agonists.

“Alkoxy” means ether-O-alkyl, where “alkyl” is as defined herein.

“Alkyl,” means saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted.

“Amino” means the group —NR1R2, where R1 and R2 are independently H, alkyl, aryl, heteroaryl, aralkyl or heteoaralkyl.

“Aminocarbonyl” means the group —NHC(O)—.

“Aminosulfonyl” means the group —NHS(O₂)—.

“Antagonist” means a material (e.g., molecule, compound, or other material) that competitively binds to a receptor at the same site on a receptor as an agonist but which does not activate the intracellular response initiated by the active form of the receptor, and can thereby inhibit the intracellular responses induced by agonists or partial agonists. Antagonists do not diminish the baseline intracellular response in the absence of an agonist or partial agonist.

“Anticipatory emesis” means a conditioned vomiting response, i.e. emesis that occurs in a subject before the subject is exposed to a substance, agent, or event (such as exposure to a chemotherapeutic agent) which has previously caused the subject to experience emesis.

“Anxiety” means a sense of apprehension and fear often marked by physical symptoms (such as sweating, tension, and increased heart rate). Anxiety can be measured in clinical and preclinical models known to those having scientific skill, knowledge and experience in these areas.

“Anxiogenic” refers to a substance, agent, event, or condition that causes anxiety.

“Aralkyl” means an alkyl group substituted with an aryl group. Suitable aralkyl groups include benzyl and the like, and these groups can be optionally substituted.

“Aryl” means aromatic groups which have at least one ring having a conjugated .pi.-electron system and includes carbocyclic aryl and biaryl, both of which can be optionally substituted. Preferred aryl groups have 6 to 10 carbon atoms.

“Binding affinity” means the affinity of a compound to bind with a receptor due to intermolecular forces between the compound and the receptor, which affect the residence time for the compound at the receptor binding site.

“Candidate compound” means a molecule, compound, or other material being screened according to the present methods. Candidate compounds can be, for example, a small molecule (e.g., a chemical compound) or a biological compound (e.g., a peptide), preferably a non-naturally occurring biological compound.

“Composition” means a material comprising at least one component; a “pharmaceutical composition” is an example of a composition.

“Compound efficacy” means a measurement of the ability of a compound to inhibit or stimulate an effect or functionality mediated by a receptor. Compound efficacy can be used to determine if a candidate compound is, for example, an agonist, antagonist, or inverse agonist.

“Constitutively activated receptor” means a receptor receptor which is capable of producing its biological response in the absence of a bound ligand. A constitutively activated receptor can be endogenous or non-endogenous.

“Constitutive receptor activation” means stabilization of a receptor in the active state by means other than binding of the receptor with its endogenous ligand or a chemical equivalent thereof.

“Contact” or “Contacting” means placing a moiety, molecule, compound, or other material (a “material”) in the same container, organism, solution, or other physical space or media as another material such that the materials interact or come into physical contact, or such that the materials at least have an opportunity to interact or make physical contact. In the present methods such contact can be in an in vitro system or an in vivo system.

“Delayed emesis” means emesis that occurs in a subject more than 24 hours after the subject is exposed to a substance, agent, or event which results in the subject experiencing emesis, or that occurs more than 24 hours after the subject contracts a condition which results in the subject experiencing emesis.

“Derivative” means a compound that is modified or partially substituted with another component. Additionally, the term “derivative” shall further encompasses compounds that can be structurally similar but can have similar or different functions.

“Emesis” means vomiting, i.e., the reflex act of ejecting the contents of the stomach through the mouth.

“Endogenous” means a material that a subject, in particular a mammal, naturally produces. Endogenous in reference to, for example and not limitation, the term “receptor,” means that which is naturally produced by a subject (for example, and not limitation, a human) or is found in the subject, for example by being introduced by a virus. By contrast, the term “non-endogenous” in this context means that which is not naturally produced by or found in a subject (for example, and not limitation, a human).

“Halo” refers to a fluoro, chloro, bromo, or iodo group

“Heteroalkyl” means carbon-containing straight-chained, branch-chained and cyclic groups, all of which can be optionally substituted, containing at least one O, N or S heteroatoms.

“Heteroaryl” means carbon-containing 5-14 membered cyclic unsaturated radicals containing one, two, three, or four O, N, or S heteroatoms and having 6, 10, or 14.pi.-electrons delocalized in one or more rings, for example, pyridine, oxazole, indole, thiazole, isoxazole, pyrazole, pyrrole, each of which can be optionally substituted as defined herein.

“Heteroaralkyl” means an alkyl group substituted with a heteroaryl group.

“Inverse agonist” means materials which bind to either the endogenous form of a receptor or to the constitutively activated form of the receptor, and which inhibit the baseline intracellular response initiated by the active form of the receptor below the normal base level of activity which is observed in the absence of agonists or partial agonists, or which decrease GTP binding to membranes. Preferably, the baseline intracellular response is inhibited in the presence of an inverse agonist by at least 30%, more preferably by at least 50%, and most preferably by at least 75%, as compared with the baseline response in the absence of the inverse agonist.

“Ligand” means a molecule specific for a receptor.

“Lower,” in reference to an alkyl or the alkyl portion of an another group including alkyl, as those terms are defined herein, means a group containing 1 to 10 carbon atoms, more typically 1 to 6 carbon atoms.

“Nausea” means a sensation of unease and discomfort in the stomach accompanied by an urge to vomit. Nausea can be measured in ways known to the art, such as through the use of a visual analog scale (VAS).

“No binding activity” means that the Ki of a candidate compound for a receptor is greater than about 10 micromolar. In this context, the “Ki” means a constant whose numerical value depends on the equilibrium between the un-dissociated and dissociated forms of a ligand or candidate compound for a receptor, whereby a higher value indicates greater dissociation, e.g., no or almost no affinity of a candidate compound for a receptor.

“Optionally substituted” means one or more substituents that are typically lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo, as those terms are defined herein.

“Treat” and “treatment” refer to a medical intervention which attenuates, prevents, or cures a medical condition, or which enhances a physiological condition, of a subject.

With respect to all chemical terms, as understood by those with skill, knowledge and experience in the field of chemistry, biology and medicine, all “groups” described herein can be optionally substituted unless such substitution is excluded.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### II. Compounds

The present compounds have the general schematic structure, {A}-L-{B}, where A is a heterocyclic ring substituted with a thio group, L is a hydrocarbyl chain attached to A through the thio group, and B is an arylpiperazine or arylpiperazine derivative.

### A. Heteroarylthio Moiety

In one embodiment of the present invention, A is a 5 atom cyclic moiety in which the five-membered ring is aromatic and has up to 1 each of a sulfur or oxygen atom and/or up to 4 nitrogen atoms, the cyclic moiety having the structure of formula (I):

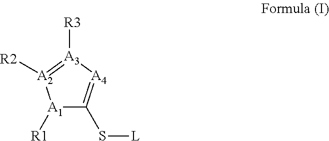

where:
(a) formula I is bonded to a hydrocarbyl linker L which is attached to moiety B;

### (b) A1 is N, O, or S;

(c) R1 is present when A1 is N and is H, alkyl, aryl, heteroaryl, aralkyl, or heteroaralkyl each of which may be optionally substituted;

### (d) When A1 is O or S, A2 is C and A3 and A4 are C or N;

### (e) When A1 is N, A2, A3 and A4 are C or N;

(f) R2 and R3 are present when A2 and A3 are C respectively;
(g) R2 and R3 are H, alkly, amino, carboxamido, sulphonamido, alkylthio, aryl, or heteroaryl each of which may be optionally substituted; and
(h) R2 and R3 may be taken together to form a six-member aromatic ring which may be optionally substituted;

One example of a heteroarylthio moiety for the moiety A is an imidazolylthio moiety of Formula (II), below:

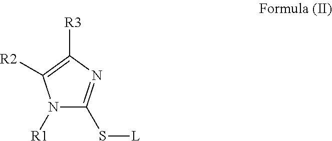

where:
(1) R₁ is H, alkyl, aralkyl, heteroaralkyl, aryl or heteroaryl each of which may be optionally substituted, and
(2) R2 and R3 are H, alkyl, aralkyl, aryl, heteroaryl or taken together to form a six-member aromatic ring as in a benzimidazole each of which may be optionally substituted.

Another example of a heteroarylthio moiety for the moiety A is a thia- or oxa-diazolylthio moiety of Formula (III), below:

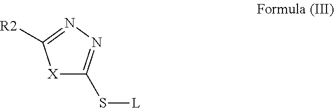

where:
R2 is alkly, aralkyl, heteroaralkyl, amino, carboxamido, sulphonamido, alkylthio, aryl, or heteroaryl each of which may be optionally substituted; and
X is O or S;

Another example of a heteroarylthio moiety for the moiety A is a tetrazolylthio moiety of Formula (IV), below:

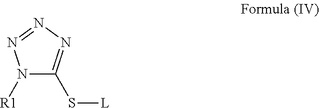

where R1 is H, alkly, aralkyl, heteroaralkyl, aryl or heteroaryl, each of which may be optionally substituted.

Another example of a heteroarylthio moiety for the moiety A is a triazolylthio moiety of Formula (V) below:

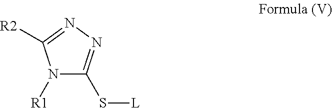

where:
R1 is H, alkly, aralkyl, heteroaralkyl, aryl or heteroaryl each of which may be optionally substituted; and
R2 is H, alkyl, aralkyl, heteroaralkyl, amino, carboxamido, sulphonamido, aryl or heteroaryl each of which may be optionally substituted.

Another example of a heteroarylthio moiety for the moiety A is a thia- or oxa-zolylthio moiety of Formula (VI), below:

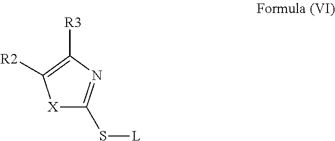

where:
R2 and R3 are independently H, alkyl, aralkyl, heteroaralkyl, aryl, heteroaryl or taken together to form a six-member aromatic ring as in a benzothiazole or benzoxazole each of which may be optionally substituted; and
X is O or S.

### B. Hydrocarbyl Linker

The linker L is preferably a hydrocarbyl moiety with the structure —(CH₂)ₘ— wherein m is an integer from 1 to 6. The linker can be substituted further with small alkyl groups. In a preferred linker, m is equal to 2, 3 or 4. The length of the linker can be varied to change the distance between the moiety A and the moiety B in the present heteroarylthio compounds.

### C. Arylpiperazine Moiety

The “B” portion of the present compounds is an arylpiperazine or derivative having the structure of Formula (VII):

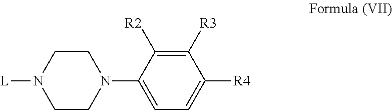

where:
(a) R2 is hydrogen, alkyl, hydroxy, halo, alkoxy, cyano, methylthio; nitro, trifluoromethyl, cycloalkyl;
(b) R3 is hydrogen, alkyl, hydroxy, halo, alkoxy, trifluoromethyl, nitro, amino, aminocarbonyl, aminosulfonyl;
(c) R2 and R3 can be taken together to form a 5 or 6 member aromatic or non-aromatic ring, which can contain from 0 to 3 heteroatoms selected from the group of N, O, or S, of which the N may be further substituted if it is secondary; and
(d) R4 is hydrogen, alkyl, halo, alkoxy, trifluoromethyl;

In one embodiment, B is an m-trifluoromethylphenylpiperazinyl moiety having the structure of Formula (VIII):

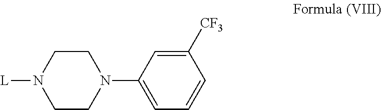

In another embodiment, B is a m-chlorophenylpiperazinyl moiety having the structure of Formula (IX):

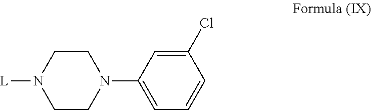

In another embodiment, B is a 1-naphthyl moiety having the structure of Formula (X):

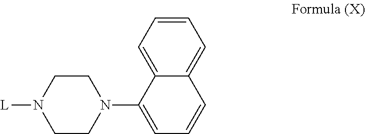

In another embodiment, B is a piperazine ring linked to a 6-member heterocyclic ring containing 1 to 2 N, having the structure of Formula (XI):

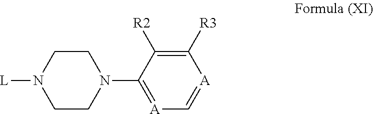

where:
(a) the 6-member heterocyclic ring can be 2-pyridyl, 4-pyridyl, or 4-pyrimidyl.
(b) R2 and R3 are taken together to form a 5 or 6 member aromatic or non-aromatic ring, which can contain from 0 to 3 heteroatoms selected from the group of N, O, or S of which the N may be further substituted if it is secondary.

In another embodiment, B is a moiety of the structure of Formula (XII):

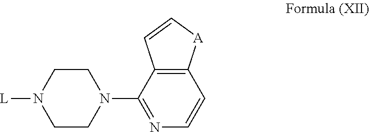

where A is O or S.

Generally, any moiety A can be combined with any linker L and any moiety B to produce one of the present compounds. The following are examples of the present compounds:
1) 1-[2-(1-Methyl-1H-imidazol-2-ylsulfanyl)ethyl]-4-(3-trifluoromethylphenyl)piperazine
2) 1-[3-(1-Methyl-1H-imidazol-2-ylsulfanyl)propyl]-4-(3-trifluoromethylphenyl)piperazine
3) 1-[2-(5-Pyridin-4-yl-[1,3,4]oxadiazol-2-ylsulfanyl)ethyl]-4-(3-trifluoromethylphenyl)piperazine
4) 1-[3-(5-Pyridin-4-yl-[1,3,4]oxadiazol-2-ylsulfanyl)propyl]-4-(3-trifluoromethylphenyl)piperazine
5) 1-[2-(1-Phenyl-1H-tetrazol-5-ylsulfanyl)ethyl]-4-(3-trifluoromethylphenyl)piperazine
6) 1-[3-(1-Phenyl-1H-tetrazol-5-ylsulfanyl)propyl]-4-(3-trifluoromethylphenyl)piperazine
7) 1-[2-(1-Methyl-1H-imidazol-2-ylsulfanyl)ethyl]-4-(3-chlorophenyl)piperazine
8) 1-[3-(1-Methyl-1H-imidazol-2-ylsulfanyl)propyl]-4-(3-chlorophenyl)piperazine
9) 1-[2-(5-Pyridin-4-yl-[1,3,4]oxadiazol-2-ylsulfanyl)ethyl]-4-(3-chlorophenyl)piperazine
10) 1-[3-(5-Pyridin-4-yl-[1,3,4]oxadiazol-2-ylsulfanyl)propyl]-4-(3-chlorophenyl)piperazine
11) 1-[2-(1-Phenyl-1H-tetrazol-5-ylsulfanyl)ethyl]-4-(3-chlorophenyl)piperazine
12) 1-[3-(1-Phenyl-1H-tetrazol-5-ylsulfanyl)propyl]-4-(3-chlorophenyl)piperazine
13) 1-[2-(1-Methyl-1H-imidazol-2-ylsulfanyl)ethyl]-4-(2,3-dichlorophenyl)piperazine
14) 1-[3-(1-Methyl-1H-imidazol-2-ylsulfanyl)propyl]-4-(2,3-dichlorophenyl)piperazine
15) 1-[2-(5-Pyridin-4-yl-[1,3,4]oxadiazol-2-ylsulfanyl)ethyl]-4-(2,3-dichlorophenyl)piperazine
16) 1-[3-(5-Pyridin-4-yl-[1,3,4]oxadiazol-2-ylsulfanyl)propyl]-4-(2,3-dichlorophenyl)piperazine
17) 1-[2-(1-Phenyl-1H-tetrazol-5-ylsulfanyl)ethyl]-4-(2,3-dichlorophenyl)piperazine
18) 1-[3-(1-Phenyl-1H-tetrazol-5-ylsulfanyl)propyl]-4-(2,3-dichlorophenyl)piperazine
19) 1-[2-(1-Methyl-1H-imidazol-2-ylsulfanyl)ethyl]-4-(3,4-dichlorophenyl)piperazine
20) 1-[3-(1-Methyl-1H-imidazol-2-ylsulfanyl)propyl]-4-(3,4-dichlorophenyl)piperazine
21) 1-[2-(5-Pyridin-4-yl-[1,3,4]oxadiazol-2-ylsulfanyl)ethyl]-4-(3,4-dichlorophenyl)piperazine
22) 1-[3-(5-Pyridin-4-yl-[1,3,4]oxadiazol-2-ylsulfanyl)propyl]-4-(3,4-dichlorophenyl)piperazine
23) 1-[2-(1-Phenyl-1H-tetrazol-5-ylsulfanyl)ethyl]-4-(3,4-dichlorophenyl)piperazine
24) 1-[3-(1-Phenyl-1H-tetrazol-5-ylsulfanyl)propyl]-4-(3,4-dichlorophenyl)piperazine
25) 1-[2-(1-Methyl-1H-imidazol-2-ylsulfanyl)ethyl]-4-(1-naphthalenyl)piperazine
26) 1-[3-(1-Methyl-1H-imidazol-2-ylsulfanyl)propyl]-4-(1-naphthalenyl)piperazine
27) 1-[2-(5-Pyridin-4-yl-[1,3,4]oxadiazol-2-ylsulfanyl)ethyl]-4-(1-naphthalenyl)piperazine
28) 1-[3-(5-Pyridin-4-yl-[1,3,4]oxadiazol-2-ylsulfanyl)propyl]-4-(1-naphthalenyl)piperazine
29) 1-[2-(1-Phenyl-1H-tetrazol-5-ylsulfanyl)ethyl]-4-(1-naphthalenyl)piperazine
30) 1-[3-(1-Phenyl-1H-tetrazol-5-ylsulfanyl)propyl]-4-(1-naphthalenyl)piperazine
31) 4-{4-[2-(1-Methyl-1H-imidazol-2-ylsulfanyl)ethyl]piperazin-1-yl}thieno[3,2-c]pyridine
32) 4-{-[3-(1-Methyl-1H-imidazol-2-ylsulfanyl)propyl]piperazin-1-yl}thieno[3,2-c]pyridine
33) 4-{4-[2-(1-Phenyl-1H-tetrazol-5-ylsulfanyl)ethyl]piperazin-1-yl}thieno[3,2-c]pyridine
34) 4-{4-[3-(1-Phenyl-1H-tetrazol-5-ylsulfanyl)propyl]piperazin-1-yl}thieno[3,2-c]pyridine
35) 4-{4-[2-(5-Pyridin-4-yl-[1,3,4]oxadiazol-2-sulfanyl)ethyl]piperazin-1-yl}thieno[3,2-c]pyridine
36) 4-{4-[3-(5-Pyridin-4-yl-[1,3,4]oxadiazol-2-sulfanyl)propyl]piperazin-1-yl}thieno[3,2-c]pyridine
37) 4-{4-[2-(1-Methyl-1H-imidazol-2-ylsulfanyl)ethyl]piperazin-1-yl}furo[3,2-c]pyridine
38) 4-{4-[3-(1-Methyl-1H-imidazol-2-ylsulfanyl)propyl]piperazin-1-yl}furo[3,2-c]pyridine
39) 4-{4-[2-(1-Phenyl-1H-tetrazol-5-ylsulfanyl)ethyl]piperazin-1-yl}furo[3,2-c]pyridine
40) 4-{4-[3-(1-Phenyl-1H-tetrazol-5-ylsulfanyl)propyl]piperazin-1-yl}furo[3,2-c]pyridine
41) 4-{4-[2-(5-Pyridin-4-yl-[1,3,4]oxadiazol-2-sulfanyl)ethyl]piperazin-1-yl}furo[3,2-c]pyridine
42) 4-{4-[3-(5-Pyridin-4-yl-[1,3,4]oxadiazol-2-sulfanyl)propyl]piperazin-1-yl}furo[3,2-c]pyridine

Preferably, the present heteroarylthio compound has a log P of from about 1 to about 4 to enhance bioavailability and central nervous system (CNS) penetration. Using this guideline, those of skill in the art can choose appropriate B moieties for a particular A moiety in order to ensure the bioavailability and CNS penetration of the present heteroarylthio compound of the present invention. For example, if a highly hydrophobic moiety A is chosen, with particularly hydrophobic substituents on the heteroaryl moiety, then a more hydrophilic moiety B is preferably used.

In general, the present heteroarylthio compounds also include salts and prodrug esters of these compounds. It is well known that organic compounds, including substituted heteroarylthios, arylpiperazines and other components of these compounds, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound herein includes such salt forms, particularly those that occur at physiological pH or at the pH of a pharmaceutical composition.

Similarly, prodrug esters can be formed by reaction of either a carboxyl or a hydroxyl group on the present heteroarylthio compound with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, halo, or other substituents, as known to those of skill in the art. The prodrug is converted into the active compound by hydrolysis of the ester linkage, typically by intracellular enzymes. Other suitable groups that can be used to form prodrug esters are known in the art.

### II. Pharmaceutical Compositions

Another aspect of the present invention is a pharmaceutical composition that comprises: (1) an effective amount of a heteroarylthio compound according to the present invention as described above and (2) a pharmaceutically acceptable excipient. A pharmaceutically acceptable excipient, including carriers, can be chosen from those generally known in the art including, but not limited to, inert solid diluents, aqueous solutions, or non-toxic organic solvents. If desired, these pharmaceutical formulations can also contain preservatives and stabilizing agents and the like, as well as minor amounts of excipients substances such as, but not limited to, a pharmaceutically acceptable excipient selected from the group consisting of wetting or emulsifying agents, pH buffering agents, human serum albumin, ion exchanger resins, antioxidants, preservatives, bacteriostatic agents, dextrose, sucrose, trehalose, maltose, alumina, lecithin, glycine, sorbic acid, propylene glycol, polyethylene glycol, protamine sulfate, sodium chloride, or potassium chloride, mineral oil, vegetable oils and combinations thereof. Those skilled in the art will appreciate that other carriers also can be used.

Liquid compositions can also contain liquid phase excipients either in addition to or to the exclusion of water. Examples of such additional liquid phases are glycerin, vegetable oils such as cottonseed oil, organic esters such as ethyl oleate, and water-oil emulsions.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solublizers, stabilizers, and preservatives. The pharmaceutical compositions with heteroarylthio compounds of the present invention can be formulated for administration by intravenous infusion, oral, topical, intraperitoneal, intravesical, transdermal, intranasal, intrarectal, intravaginal, intramuscular, intradermal, subcutaneous and intrathecal routes.

Pharmacuetical formulations of the present heteroarylthio compounds can be presented in unit-dose or multi-dose sealed containers, in physical forms such as ampules or vials. The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

### III. Screening

In order to identify particularly preferred candidate compounds, the compounds can be screened using assay techniques. Assay techniques for screening of candidate compounds, are well known to those of skill in the art.

With respect to receptor-based screening assays, candidate compounds can be initially screened to determine if the compounds bind with the receptor using competitive binding assays, that is, assays designed to typically measure the ability of the candidate compound to compete with the receptor's ligand for binding to the receptor. The compound efficacy of a candidate compound is also preferably determined. Compound efficacy is often used to determine what impact the candidate compound has on the activity of the receptor to effectuate a desired biological outcome. Identifying the compound efficacy of a candidate compound is useful in identifying the type of receptor-activity that the candidate compound can have, for example, as an agonist, antagonist, inverse agonist.

Once candidate compounds are screened to select for those compounds that have preferred characteristics, such compound(s) can be tested in animal models to assess the attributes of the compound(s) in a living animal. Those with scientific skill, knowledge and experience in this field understand the methods and procedures for the testing of candidate compounds in animal models of conditions, diseases or disorders.

Binding affinity assays often rely upon the binding affinity of a compound, which can be expressed as the Ki of the candidate compound for that receptor. In terms of compound efficacy, a multitude of assays exist which measure different aspects of compound efficacy. For example, in the context of screening assays for GPCRs, those with scientific skill, knowledge and experience understand that assays exist for cyclic-AMP production which measure the ability of a candidate compound to impact the production of cyclic-AMP as an indication of the biological impact that the candidate compound has. However, other similar measurements can be made with different outcome measures, for example, calcium ion mobilization.

Compound efficacy can be measured in terms of EC50, that is, the molar concentration of the candidate compound which produces 50% of the maximum possible effective response for that compound. While EC₅₀ is the measurement used to determine compound efficacy, there are also other ways to determine this value. These can be based on products produced by the receptor when contacted with a candidate compound. The produced product generates a signal, and this signal is measured, and most often compared to the signal measured based upon binding of the ligand to the receptor. An example of one such product measured in GPCR assays is cyclic-AMP, as is well known to those of skill in the art.

### IV. Treatments

Neurological conditions, including psychiatric conditions, can be treated by administering therapeutically effective amounts of the present compounds and/or pharmaceutical compositions. These compounds can be used as anti-psychotic compounds and administered to treat psychiatric disorders such as depression, anxiety including post traumatic stress syndrome, schizophrenia, schizoaffective disorders, bipolar disorders, sexual dysfunction, mood swings, sleep disorders, anorexia, bulimia, manic depression, obsessive compulsive disorders, delusional post-partum depression, post-partum psychosis, pre-menstrual syndrome, drug abuse associated psychoses and combinations thereof. The present compounds can also be used to enhance cognitive function and to treat neuroregenerative disorders with cognitive deterioration such as Parkinson's disease, Huntington's disease, Alzheimer's disease, dementia associated with aging, and exposure to toxic chemical agents such as soman and saran.

Emotional, mood swings and cognitive disorders related to psychiatric disturbances that are expressed as sleep disorders, anorexia, bulimia, post-partum depression, post-partum psychosis, pre-menstrual syndrome, manic depression, obsessive compulsive disorders, and delusional disorders can also be treated using the present compounds and pharmaceutical compositions. Other emotional disturbances that can be effectively treated include those related to substance abuse. For example, the present pharmaceutical compositions can be used to prevent drug dependence or tolerance including that produced by nicotine, opioids such as morphine, cocaine and barbiturates such as diaxepam. Furthermore, the pharmaceutical compositions of the present invention can be useful in preventing or treating emotional and cognitive disturbances or psychoses associated with drug withdrawal or cessation tolerance including that produced by nicotine, opioids such as morphine, cocaine and barbiturates such as diaxepam.

Cognitive and other neurological disorders that can be effectively treated using the present compounds and pharmaceutical compositions include conditions such as, but not limited to, neurosensory diseases and injury, Parkinson's disease and other movement disorders such as dystonia, Wilson's disease, inherited ataxias, Tourette syndrome cerebral palsy, encephalopathies. Other cognitive conditions that can be treated include cognitive and attention deficit disorders associated with acquired immunodeficiency syndrome (AIDS), dementia, ischemic stroke, chemical exposure, and cardiac bypass associated cognitive defects.

Pain can be effectively treated with the compounds and pharmaceutical compositions of the present invention by administering an effective amount of these compounds and/or compositions to a patient in need thereof, in particular by administering an analgesic dosage of these compositions. Among the different types of pain that can be treated with the present compounds are acute pain, chronic pain, nociceptive pain (i.e., pain associated with pain transmission through intact nerve endings), and neuropathic pain (caused by nervous system dysfunction and characterized by burning, shooting, and tingling pain, associated with allodynia, hyperpathia, paresthesias and dysesthesias). Conditions which can involve acute pain include headache, arthritis, simple muscle strain, and dysmenorrhea. Nociceptive pain can include, e.g., post-operative pain, cluster headaches, dental pain, surgical pain, pain resulting from burns, post partum pain, angina pain, genitourinary tract related pain, cystitis, pain associated with arthritis, AIDS, chronic back pain, visceral organ pain, gastroesophageal reflux, peptic ulcers, infectious gastritis, inflammatory bowel disorders, migraine headaches, tension headaches, fibromyalgia, nerve root compression such as sciatica, trigeminal neuralgia, central pain, bone injury pain, pain during labor and delivery, muscle strain, alcoholism, herpetic neuralgia, phantom limb pain, and dysmenorrheal pain. Conditions involving neuropathic pain include chronic lower back pain, pain associated with arthritis, cancer-associated pain, herpes neuralgia, phantom limb pain, central pain, opioid resistant neuropathic pain, bone injury pain, and pain during labor and delivery. Relief from pain-induced psychiatric disorders such as anxiety, depression and/or severe mood changes as well as emetic responses related to pain and its treatment can also be provided with the present compounds and compositions.

An additional use of the present compounds and/or pharmaceutical compositions is in stimulating neurogenesis, neuronal regeneration or axo-dendritic complexity in the central and peripheral nervous systems. This is accomplished through the step of administering an effective amount of a compound according to the present invention to a subject in need thereof. Such neuroregenerative effects are believed to be the result of the 5-HT1A receptor agonist activity of the compounds. Neurodegenerative conditions that can be treated can be genetic, spontaneous or iatrogenic, including, but not limited to, stroke, spinal cord injury amyotrophic lateral sclerosis, perinatal hypoxia, ocular damage and retinopathy, ocular nerve degeneration, hearing loss, restless leg syndrome, Gulf War Syndrome and Tourette's syndrome.

The compounds of the present invention can also be used to treat peripheral neuropathies. Examples of diseases associated with peripheral neuropathies include, but are not limed to, acromegaly, hypothyroidism, AIDS, leprosy, Lyme disease, systemic lupus erythematosus, rheumatoid arthritis, Sjogren's Syndrome, periarteritis nodosa, Wegener's granulomatosis, cranial arteritis, sarcoidosis, diabetes, vitamin B12 deficiency, cancer, Gulf War Syndrome and alcoholism. Examples of drug therapies associated with peripheral neuropathies include, but are not limed to oncolytic drugs such as a vinca alkaloid, platinum derivatives such as cisplatin, paclitaxel, suramin, altretamine, carboplatin, chlorambucil, cytarabine, dacarbazine, docetaxel, etoposide, fludarabine, ifosfamide with mesna, tamoxifen, teniposide, or thioguanine.

In one embodiment, the compounds of the present application can be combined with other analgesics to form a pharmaceutical composition, in order to lower the dose of the present compounds required to relieve pain and/or to achieve a synergistic reduction in pain experienced by a patient. Other analgesics which can be co-administered with the present compounds (either at the same time or at different times) include aspirin, ibuprophen, acetaminophen, opiates, acetaminophen combined with codeine, indomethacin, tricyclic antidepressants, anticonvulsants, serotonin reuptake inhibitors, mixed serotonin-norepinephrine reuptake inhibitors, serotonin receptor agonists and antagonists, cholinergic analgesics, adrenergic agents, and neurokinin antagonists. Other analgesics can be found, for example, in the Merck Manual, 16th Ed. (1992) p. 1409.

In a preferred embodiment, a compound or composition as described above can be used to treat emesis. Candidate compounds can be screened and further tested in animals to further elucidate the opportunity for such compounds to function as anti-emetic compounds that have reduced or no anxiety side effects. Such compounds can then be further evaluated in humans. One or more of the candidate compounds that meet the criteria disclosed herein can then be provided as composition(s) to a mammal such as a human. Such composition(s) are beneficial in the treatment of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. The use of such compositions is also of benefit in the therapy of emesis induced by radiation, for example during the treatment of cancer, and in the treatment of post-operative nausea and vomiting. The use of such compositions is also beneficial in the therapy of emesis induced by antineoplastic (cytotoxic) agents including those routinely used in cancer chemotherapy, and emesis induced by other pharmacological agents. Further, the use of such compositions can also be used in the therapy of acute, delayed or anticipatory emesis from an unknown cause.

The effects of nerve agent exposure can also be prevented or ameliorated by administering therapeutically effective amounts of one or more of the present compounds and/or pharmaceutical compositions to a patient in need thereof. Such agents include organophosphate anticholinesterase agents such as tabun (Ethyl N,N-dimethylphosphoramidocyanidate, also referred to as GA), sarin (O-Isopropyl methylphosphonofluoridate, also referred to as GB), soman (O-Pinacolyl methylphosphonofluoridate, also referred to as GD), and VX (O-ethyl-S-[2(diisopropylamino)ethyl]methylphosphonothiolate). The present compounds and/or compositions are administered to a patient in a quantity sufficient to treat or prevent the symptoms and/or the underlying etiology associated with nerve agent exposure in the patient. The present compounds can also be administered in combination with other agents known to be useful in the treatment of nerve agent exposure, such as atropine sulfate, diazepam, and pralidoxime (2-PAM), either in physical combination or in combined therapy through the administration of the present compounds and agents in succession (in any order).

Administration of the present compounds and compositions can begin immediately following exposure to an organophosphate nerve agent, preferably within the first hour following exposure, and more preferably within one to five minutes. Administration of the compositions and compounds can alternatively begin prior to an anticipated exposure (such as impending combat), in order to prevent or reduce the impact of subsequent exposure. The present invention thus includes the use of the present compounds and/or a pharmaceutical composition comprising such compounds to prevent and/or treat exposure to a nerve agent.

Depending upon the particular needs of the individual subject involved, the compounds of the present invention can be administered in various doses to provide effective treatment concentrations based upon the teachings of the present invention. Factors such as the activity of the selected compounds, the physiological characteristics of the subject, the extent or nature of the subject's disease or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired.

An effective amount of any embodiment of the present invention is determined using methods known to pharmacologists and clinicians having ordinary skill in the art. For example, an animal model can be used to determine applicable dosages for a patient. As known to those of skill in the art, a very low dose of a compound, i.e. one found to be minimally toxic in animals (e.g., 1/10×LD10 in mice), can first be administered to a patient, and if that dose is found to be safe, the patient can be treated at a higher dose. In one example, a therapeutically effective amount of one of the present compounds for treating nerve agent exposure can then be determined by administering increasing amounts of such compound to a patient suffering from such exposure until such time as the patient's symptoms are observed or are reported by the patient to be diminished or eliminated.

In a preferred embodiment, the present compounds and compositions have a therapeutic index of approximately 2 or greater. The therapeutic index is determined by dividing the dose at which adverse side effects occur by the dose at which efficacy for the condition is determined. A therapeutic index is preferably determined through the testing of a number of subjects. Another measure of therapeutic index is the lethal dose of a drug for 50% of a population (LD₅₀, in a pre-clinical model) divided by the minimum effective dose for 50% of the population (ED₅₀).

In another example, a pain relieving effective amount can be determined subjectively by administering increasing amounts of the pharmaceutical compositions of the present invention until such time the patient being treated reports diminishment in pain sensations. Blood levels of the drug can be determined using routine biological and chemical assays and these blood levels can be matched to the route of administration. The blood level and route of administration giving the most desirable level of pain relief can then be used to establish an “effective amount” of the pharmaceutical composition for treating the pain under study. This same method of titrating a pharmaceutical composition in parallel with administration route can be used to ascertain an “effective amount” of the pharmaceutical compositions of the present invention for treating any and all psychiatric or neurological disorders described herein. In addition, animal models as described below can be used to determine applicable dosages for a particular condition.

Exemplary dosages in accordance with the teachings of the present invention for these compounds range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the compound is being administered, the response to treatment, the type and quantity of other medications being given to the patient that might interact with the compound, either potentiating it or inhibiting it, and other pharmacokinetic considerations such as liver and kidney function.

### V. Synthesis

The present compounds can be produced by substituting one of the heteroarylthio moieties described above with a linker that in turn is linked to an arylpiperazine moiety. This route can comprise the steps of (1) synthesizing an appropriately substituted heteroarylthio moiety linked to an aliphatic linker in which the linker is terminated with a halogen and (2) reacting the halogen intermediate with the arylpiperazine to produce the final product. Alternatively, an appropriately substituted arylpiperazine moiety linked to an aliphatic linker, in which the linker is terminated with a halogen, can be produced and a halogen intermediate can be reacted with a heteroarylmercaptan to produce the final product.

The present heteroarylthio compounds of the present invention can be synthesized, for example, by a dihalide substitution reaction. Suitable substitution reactions are described, e.g., in M. B. Smith & J. March, “March's Advanced Organic Chemistry: Reactions, Mechanisms, and Structure” (5^{th} ed., Wiley-Interscience, New York, 2001).

The following representative methods for synthesizing exemplary embodiments of the present invention are merely intended as examples. Persons with skill, knowledge and experience in the areas of medicinal and/or organic chemistry will understand that other starting materials, intermediates, reaction conditions are possible. The present examples represent but one particular method for synthesizing the composite, biologically active molecules of the present invention. Furthermore, it is understood that various salts of these compounds are also easily made and these salts can have biological activity similar or exactly equivalent to the parent compound. Generally, these salts have chloride or bromide as the anion. However, other anions can be used and are considered within the scope of the present invention.

### Synthesis of 1-(2-chloroethyl)-4-(3-(trifluoromethyl)phenyl)piperazine

4.6 g 1-(3-trifluoromethyl)phenyl)piperazine (20 mm, 3.76 ml) and 0.8 g powdered NaOH (20 mm) were added into 30 ml DMSO. The mixture was stirred for approx. 10 min. 4.3 g 1-bromo-2-chloro ethane (30 mm, 2.5 ml) was then added thereto. The mixture was allowed to react for 24 hours, followed by TLC analysis (silica gel, ethyl acetate:dichloromethane 1:1) which indicated that the reaction was essentially complete. The reaction mixture was poured into ice water (180 ml) and a yellow liquid oiled out. The oil was dissolved with dichloromethane (30 ml) and the ice water-reaction mixture was extracted with further dichloromethane (30 ml). The organics were combined, dried over Na₂SO₄, and allowed to completely dry with a rotovap. The material was then purified on a Chromatotron (1000 micron silica gel, dichloromethane) to yield the final product as an oil.

### Synthesis of 1-{2-[(1-methyl-1H-imidazol-2-yl)thio]ethyl}-4-[3-(trifluoromethyl)phenyl]piperazine (Compound I)

57 mg (0.5 mm) 2-mercapto-1-methylimidazole, 146 mg (0.5 mm) 1-(2-chloroethyl)-4-(3-(trifluoromethyl)phenyl)piperazine and 69 mg K₂CO₃ were dissolved in 2 ml acetonitrile. The reaction was left stifling at room temperature for 7 days and the reaction went to completion. 9 ml water was then added, and an oil appeared. The oil was dissolved in ethyl acetate, washed with water, dried over Na₂SO₄, and allowed to completely dry with a rotovap. The material was then purified on a Chromatotron (1000 micron silica gel, ethyl acetate:dichloromethane 3:1) to yield the final product as a yellowish solid. HPLC analysis indicated a purity of 96.4%. MS analysis indicated an M+1 molecular ion m/z of 371.1, calculated 371.1.

### Synthesis of 1-[2-(5-pyridin-4-yl-[1,3,4]oxadiazol-2-ylsulfanyl)ethyl]-4-(3-trifluoromethylphenyl)piperazine (Compound 3)

90 mg (0.5 mm) 5-(4-pyridyl)-1,3,4-oxadiazole-2-thiol, 146 mg (0.5 mm) 1-(2-chloroethyl)-4-(3-(trifluoromethyl)phenyl)piperazine, and 69 mg K₂CO₃ in were dissolved in 2 ml acetonitrile. The mixture was heated to 60° C. for 5 hours and the reaction went to completion. 9 ml water was then added, and an oil appeared. The oil was dissolved in ethyl acetate, washed with water, dried over Na₂SO₄, and allowed to completely dry with a rotovap. The material was then purified on a Chromatotron (1000 micron silica gel, ethyl acetate:dichloromethane 3:1) to yield the final product as a yellowish solid. HPLC analysis indicated a purity of 99.7%. MS analysis indicated an M+1 molecular ion m/z of 436.1, calculated 436.1.

### Synthesis of 1-{3-[(1-methyl-1H-imidazol-2-yl)thio]propyl}-4-[3-chlorophenyl]piperazine (Compound 8)

100 mg (0.87 mm) 2-mercapto-1-methylimidazole, 239 mg (0.87 mm) 1-(3-chloropropyl)-4-(3-chlorophenyl)piperazine and 120 mg K₂CO₃ were dissolved in 4 ml acetonitrile. The mixture was heated to 60° C. for 5 hours and the reaction went to completion. 12 ml water was then added, and an oily solid appeared. The oily solid was dissolved in ethyl acetate, washed with water, dried over Na₂SO₄, and allowed to completely dry with a rotovap. The material was then purified on a Chromatotron (1000 micron silica gel, ethyl acetate:dichloromethane 3:1) to yield the final product as a yellowish solid. HPLC analysis indicated a purity of 98.7%. MS analysis indicated an M+1 molecular ion m/z of 350.9, calculated 350.9.

### Synthesis of 1-[2-(5-pyridin-4-yl-[1,3,4]oxadiazol-2-ylsulfanyl)ethyl]-4-(3-trifluoromethylphenyl)piperazine (Compound 10)

131 mg (0.73 mm) 5-(4-Pyridyl)-1,3,4-oxadiazole-2-thiol, 200 mg (0.73 mm) 1-(3-chloropropyl)-4-(3-chlorophenyl)piperazine, and 101 mg K₂CO₃ in were dissolved in 4 ml acetonitrile. The mixture was heated to 60° C. for 4 hours and the reaction went to completion. 10 ml water was then added, and an oil appeared. The oil was dissolved in ethyl acetate, washed with water, dried over Na₂SO₄, and allowed to completely dry with a rotovap. The material was then purified on a Chromatotron (1000 micron silica gel, ethyl acetate:dichloromethane 3:1) to yield the final product as a yellowish solid. HPLC analysis indicated a purity of 99.1%. MS analysis indicated an M+1 molecular ion m/z of 415.9, calculated 415.9.

### Synthesis of 1-[3-(1-phenyl-1H-tetrazol-5-ylsulfanyl)propyl]-4-(3-chlorophenyl)piperazine (Compound 12)

120 mg (0.67 mm) 1-Phenyl-1H-tetrazole-5-thiol, 184 mg (0.67 mm) 1-(3-chloropropyl)-4-(3-chlorophenyl)piperazine, and 93 mg K₂CO₃ in were dissolved in 4 ml acetonitrile. The mixture was heated to 60° C. for 6 hours and the reaction went to completion. 10 ml water was then added, and an oil appeared. The oil was dissolved in ethyl acetate, washed with water, dried over Na₂SO₄, and allowed to completely dry with a rotovap. The material was then purified on a Chromatotron (1000 micron silica gel, ethyl acetate:dichloromethane 4:1) to yield the final product as a yellowish solid. HPLC analysis indicated a purity of 99.2%. MS analysis indicated an M+1 molecular ion m/z of 415.0, calculated 415.0.

### Synthesis of 4-{4-[3-(1-methyl-1H-imidazol-2-ylsulfanyl)propyl]piperazin-1-yl}furo[3,2-c]pyridine (Compound 38)

80 mg (0.7 mm) 2-mercapto-1-methylimidazole, 196 mg (0.7 mm) 4-[4-(3-chloropropyl)piperazin-1-yl]furo[3,2-c]pyridine and 97 mg K₂CO₃ were dissolved in 3 ml acetonitrile. The mixture was heated to 60° C. for 5 hours and the reaction went to completion. 10 ml water was then added, and an oily solid appeared. The oily solid was dissolved in ethyl acetate, washed with water, dried over Na₂SO₄, and allowed to completely dry with a rotovap. The material was then purified on a Chromatotron (1000 micron silica gel, ethyl acetate:dichloromethane 2:1) to yield the final product as a yellowish solid. HPLC analysis indicated a purity of 98.9%. MS analysis indicated an M+1 molecular ion m/z of 357.5, calculated 357.5.

### Synthesis of 4-{4-[3-(1-phenyl-1H-tetrazol-5-ylsulfanyl)propyl]piperazin-1-yl}furo[3,2-c]pyridine (Compound 40)

107 mg (0.6 mm) 1-Phenyl-1H-tetrazole-5-thiol, 168 mg (0.6 mm) 4-[4-(3-chloropropyl)piperazin-1-yl]furo[3,2-c]pyridine, and 83 mg K₂CO₃ in were dissolved in 3 ml acetonitrile. The mixture was heated to 60° C. for 6 hours and the reaction went to completion. 10 ml water was then added, and an oil appeared. The oil was dissolved in ethyl acetate, washed with water, dried over Na₂SO₄, and allowed to completely dry with a rotovap. The material was then purified on a Chromatotron (1000 micron silica gel, ethyl acetate:dichloromethane 3:1) to yield the final product as a yellowish solid. HPLC analysis indicated a purity of 98.8%. MS analysis indicated an M+1 molecular ion m/z of 421.5, calculated 421.5.

### Synthesis of 4-{4-[3-(5-pyridin-4-yl-[1,3,4]oxadiazol-2-sulfanyl)propyl]piperazin-1-yl}furo[3,2-c]pyridine (Compound 42)

134 mg (0.75 mm) 5-(4-Pyridyl)-1,3,4-oxadiazole-2-thiol, 210 mg (0.75 mm) 4-[4-(3-chloropropyl)piperazin-1-yl]furo[3,2-c]pyridine, and 104 mg K₂CO₃ in were dissolved in 4 ml acetonitrile. The mixture was heated to 60° C. for 6 hours and the reaction went to completion. 12 ml water was then added, and an oily solid appeared. The oily solid was dissolved in ethyl acetate, washed with water, dried over Na₂SO₄, and allowed to completely dry with a rotovap. The material was then purified on a Chromatotron (1000 micron silica gel, ethyl acetate:dichloromethane 2:1) to yield the final product as a yellowish solid. HPLC analysis indicated a purity of 99.3%. MS analysis indicated an M+1 molecular ion m/z of 422.5, calculated 422.5.

### VI. Examples

### Example 1

### Receptor Binding

A set of compounds were tested for their ability to inhibit binding of a binding ligand to the 5HT1A, 5HT1D, and 5HT7 receptors in a competitive assay. The tested compounds were placed in the assay with either radiolabeled OH-DPAT (which binds the 5HT1A receptor), radiolabeled serotonin (which binds the 5HT1D receptor), or radiolabeled LSD (lysergic acid diethylamide, which binds the 5HT7 receptor).

The results of the foregoing tests are shown in Table 1 below. All compounds were found to be strong inhibitors of 5HT1A binding, with varying degrees of binding to 5HT1D and 5HT7 receptors as well.

TABLE 1
Percent inhibition of receptor binding at 100 nM

| | 5HT1A | 5HT1D | 5HT7 |
|---|---|---|---|
| | Recep- | Recep- | Recep- |
| | tor | tor | tor |
| Compound | Binding | Binding | Binding |
| 1-[2-(5-Pyridin-4-yl-[1,3,4]oxadiazol-2- | 92% | 10% | 25% |
| ylsulfanyl)ethyl]-4-(3- | | | |
| trifluoromethylphenyl) piperazine | | | |
| (Example 3) | | | |
| 1-[2-(1-Methyl-1h-imidazol-2- | 87% | 52% | 63% |
| ylsulfanyl)ethyl]-4-(3- | | | |
| trifluoromethylphenyl)piperazine | | | |
| (Example 1) | | | |
| 1-[3-(5-Pyridin-4-yl-[1,3,4]oxadiazol-2- | 97% | 51% | 8% |
| ylsulfanyl)propyl]-4-(3-chlorophenyl) | | | |
| piperazine | | | |
| (Example 10) | | | |
| 1-[3-(1-Phenyl-1H-tetrazol-5- | 75% | 6% | 43% |
| ylsulfanyl)propyl]-4-(3- | | | |
| chlorophenyl)piperazine | | | |
| (Example 12) | | | |
| 1-[3-(1-Methyl-1H-imidazol-2- | 94% | 50% | 82% |
| ylsulfanyl)propyl]-4-(3- | | | |
| chlorophenyl)piperazine | | | |
| (Example 8) | | | |
| 4-{4-[3-(5-Pyridin-4-yl-[1,3,4]oxadiazol- | 96% | 65% | 21% |
| 2-sulfanyl)propyl]piperazin-1-yl}furo[3,2- | | | |
| c] pyridine | | | |
| (Example 42) | | | |
| 4-{4-[3-(1-Phenyl-1H-tetrazol-5- | 97% | 38% | 67% |
| ylsulfanyl)propyl]piperazin-1-yl}furo[3,2- | | | |
| c]pyridine | | | |
| (Example 40) | | | |
| 4-{4-[3-(1-Methyl-1H-imidazol-2- | 71% | (not | 81% |
| ylsulfanyl)propyl]piperazin-1-yl}furo[3,2- | | tested) | |
| c]pyridine | | | |
| (Example 38) | | | |

### Example 2

### Anti-Psychotic Activity

The Condition Avoidance Responding (CAR, active avoidance) model is used as a test for antipsychotic activity. The disruption of avoidance (increased latency) without disruption of escape (extrapyramidal motor function) is a clinical predictor of compounds with antipsychotic activity. The compound of example 10 above (1-[3-(5-Pyridin-4-yl-[1,3,4]oxadiazol-2-ylsulfanyl)propyl]-4-(3-chlorophenyl)piperazine) was evaluated in this model as follows.

Training of animals (mice) consisted of 20 trials with variable inter-trial intervals (trained to 80% Avoidance Criteria). After a one-minute acclimation period, the house light and an acoustic 90 dB tone (conditioned stimuli) were presented. A response (crossing to a dark compartment) within 5 seconds ended the trial and the trial was recorded as avoidance response (CAR). If the mouse did not respond within 5 seconds, foot shock (0.8 mA) was presented, and the response (moving to the dark chamber) during the shock was recorded as an escape response. To avoid shock, animals learn to move from the lighted side of the chamber to the dark side when the cue is presented (avoidance) or moved when the shock is administered (escape). Vehicle or test compounds were administered subcutaneously 30 minutes before the test session.

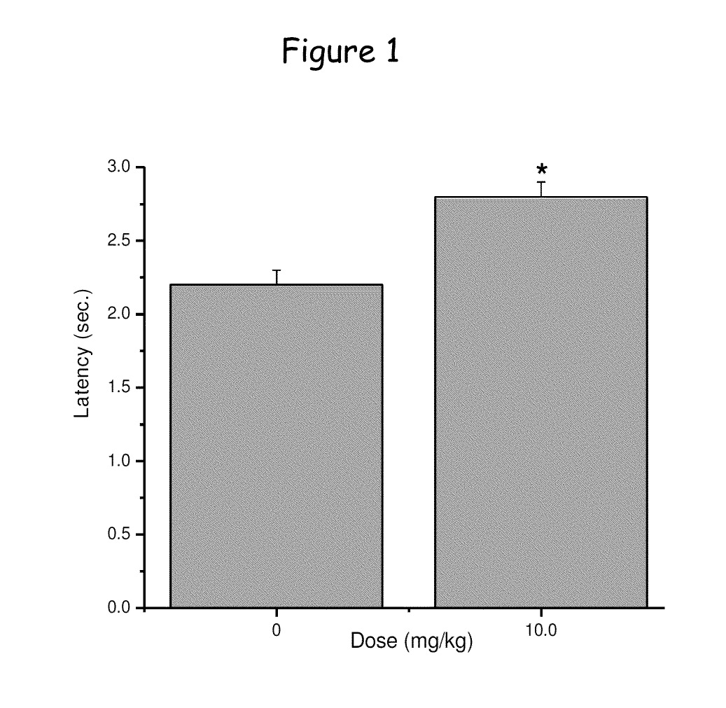

C57 male mice were tested 24 hours after being trained as described above. FIG. 1 shows results of this test. At a dose of 10 mg/kg, the test compound resulted in a statistically significant increase in latency, and thus an indication of antipsychotic activity.

### Example 2

### Memory

The Condition Avoidance Responding (CAR, active avoidance) model, although primarily a test for detecting anti-psychotic activity, includes an element of training and learning (acquisition of memory), and thus can also be used as a model for testing a compound's effects on learning (acquisition of information) and memory (retention of information). A decrease in the latency of an animal's response indicates an enhancement of an animal's memory of the stimulus. The compound of example 12 above (1-[3-(1-Phenyl-1H-tetrazol-5-ylsulfanyl)propyl]-4-(3-chlorophenyl)piperazine) was evaluated in this model as follows.

Training of animals (mice) consisted of 20 trials with variable inter-trial intervals (trained to 80% Avoidance Criteria). After a one-minute acclimation period, the house light and an acoustic 90 dB tone (conditioned stimuli) were presented. A response (crossing to a dark compartment) within 5 seconds ended the trial and the trial was recorded as avoidance response (CAR). If the mouse did not respond within 5 seconds, foot shock (0.8 mA) was presented, and the response (moving to the dark chamber) during the shock was recorded as an escape response. To avoid shock, animals learn to move from the lighted side of the chamber to the dark side when the cue is presented (avoidance) or moved when the shock is administered (escape). Vehicle or test compounds were administered subcutaneously 30 minutes before the test session.

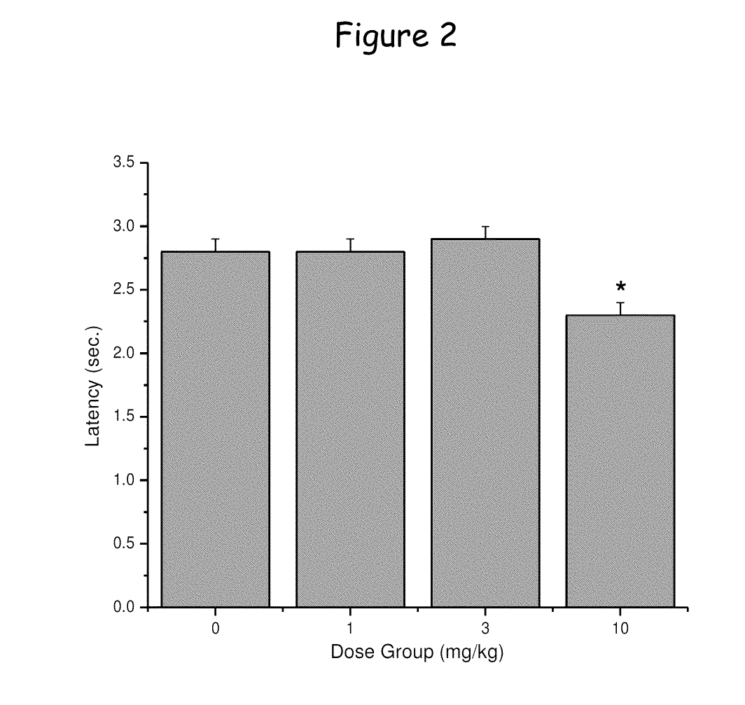

C57 male mice were tested 24 hours after being trained as described above. FIG. 2 shows results from this test. At a dose of 10 mg/kg, the test compound resulted in a statistically significant decrease in latency, thus indicating an enhancement of the animals' memory of the stimulus.

Preferred embodiments of our invention are described herein, including the best mode known to the inventors for carrying out the invention. Of course, variations on those preferred embodiments will become apparent to those having skill, knowledge and experience in the field upon reading the foregoing description. Recitation of value ranges herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

1. A heteroarylthio compound having the following formula: 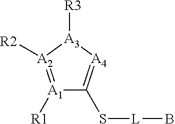
where:
(a) A1 is N, O, or S;
(b) R1 is present when A1 is N and is H, alkyl, aryl, heteroaryl, aralkyl, or heteroaralkyl, each of which may be optionally substituted;
(c) when A1 is O or S, A2 is C and A3 and A4 are C or N;
(d) when A1 is N, A2, A3 and A4 are C or N;
(e) R2 and R3 are present when A2 and A3 are C respectively;
(f) R2 and R3 are H, alkly, amino, carboxamido, sulphonamido, alkylthio, aryl, or heteroaryl, each of which may be optionally substituted; and
(g) R2 and R3 can be taken together to form a six-member aromatic ring which may be optionally substituted;
(h) L is (CH₂)ₘ, wherein m is an integer from 1 to 6; and
(i) B has the following formula: 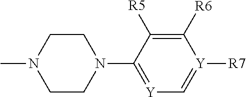
where:
(1) Y is C or N;
(2) R5 is hydrogen, alkyl, hydroxy, halo, alkoxy, cyano, methylthio; nitro, trifluoromethyl, or cycloalkyl;
(3) R6 is hydrogen, alkyl, hydroxy, halo, alkoxy, trifluoromethyl, nitro, amino, aminocarbonyl, or aminosulfonyl;
(4) R5 and R6 can be taken together to form a 5 or 6 member aromatic or non-aromatic ring, which can contain from 0 to 3 heteroatoms selected from the group of N, O, or S, of which the N may be further substituted if it is secondary; and
(5) R7 is hydrogen, alkyl, halo, alkoxy, or trifluoromethyl, or is absent if Y is N.

2. The heteroarylthio compound of claim 1, wherein the compound has the following formula: 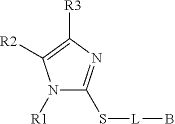
where:
(a) R₁ is H, alkyl, aralkyl, heteroaralkyl, aryl or heteroaryl, each of alkyl, aralkyl, heteroaralkyl, aryl or heteroaryl which may be optionally substituted, and
(b) R₂ and R₃ are each independently selected from H, alkyl, aralkyl, aryl, heteroaryl or R₂ and R₃ may be taken together to form a six-member aromatic ring, each of which may be optionally substituted.

3. The heteroarylthio compound of claim 1, wherein the compound has the following formula: 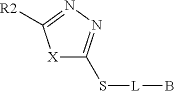
where:
(a) R₂ is alkly, aralkyl, heteroaralkyl, amino, carboxamido, sulphonamido, alkylthio, aryl, or heteroaryl, each of alkly, aralkyl, heteroaralkyl, amino, carboxamido, sulphonamido, alkylthio, aryl, or heteroaryl which may be optionally substituted; and
(b) B is O or S.

4. The heteroarylthio compound of claim 1, wherein the compound has the following formula: 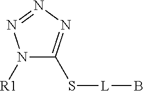
wherein R₁ is H, alkly, aralkyl, heteroaralkyl, aryl or heteroaryl, each of alkly, aralkyl, heteroaralkyl, aryl or heteroaryl which may be optionally substituted.

5. The heteroarylthio compound of claim 1, wherein the compound has the following formula: 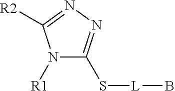
where:
(a) R₁ is H, alkly, aralkyl, heteroaralkyl, aryl or heteroaryl, each of the alkly, aralkyl, heteroaralkyl, aryl or heteroaryl which may be optionally substituted; and
(b) R₂ is H, alkyl, aralkyl, heteroaralkyl, amino, carboxamido, sulphonamido, aryl or heteroaryl, each of alkyl, aralkyl, heteroaralkyl, amino, carboxamido, sulphonamido, aryl or heteroaryl which may be optionally substituted.

6. The heteroarylthio compound of claim 1, wherein the compound has the following formula: 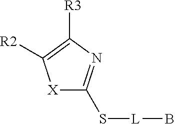
where:
(a) R₂ and R₃ are independently H, alkyl, aralkyl, heteroaralkyl, aryl, heteroaryl or R₂ and R₃ are taken together to form a six-member aromatic ring, each of which may be optionally substituted; and
(b) X is O or S.

7. The heteroarylthio compound of claim 1, wherein m is selected from the group consisting of 2, 3, and 4.

8. The heteroarylthio compound of claim 1, wherein L is substituted with alkyl groups.

9. The heteroarylthio compound of claim 1, wherein B is a m-trifluoromethylphenylpiperazinyl moiety having the following formula:

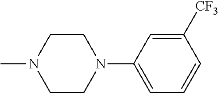 [SMILES: CN1CCN(c2cccc(C(F)(F)F)c2)CC1]

10. The heteroarylthio compound of claim 1, wherein B is a m-chlorophenylpiperazinyl moiety having the following formula:

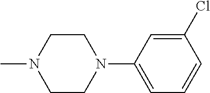

11. The heteroarylthio compound of claim 1, wherein B is a 1-naphthyl moiety having the following formula:

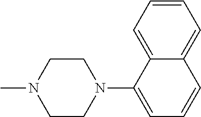 [SMILES: CN1CCN(c2cccc3ccccc23)CC1]

12. The heteroarylthio compound of claim 1, wherein B is a moiety having the following formula: 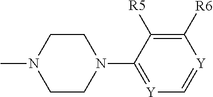
where:
(a) the 6-member heterocyclic ring can be 2-pyridyl, 4-pyridyl, or 4-pyrimidyl; and
(b) R5 and R6 can be taken together to form a 5 or 6 member aromatic or non-aromatic ring, which can contain from 0 to 3 heteroatoms selected from the group of N, O, or S, of which the N may be further substituted if it is secondary

13. The heteroarylthio compound of claim 1, wherein B is a moiety having the following formula: 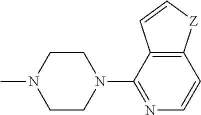
where Z is O or S.

14. The heteroarylthio compound of claim 1, wherein the compound is selected from the group consisting of:
1-{2-[(1-methyl-1H-imidazol-2-yl)thio]ethyl}-4-[3-(trifluoromethyl)phenyl]piperazine;
1-[2-(5-pyridin-4-yl-[1,3,4]oxadiazol-2-ylsulfanyl)ethyl]-4-(3-trifluoromethylphenyl)piperazine;
1-{3-[(1-methyl-1H-imidazol-2-yl)thio]propyl}-4-[3-chlorophenyl]piperazine;
1-[3-(1-phenyl-1H-tetrazol-5-ylsulfanyl)propyl]-4-(3-chlorophenyl)piperazine; and
1-[2-(5-pyridin-4-yl-[1,3,4]oxadiazol-2-ylsulfanyl)ethyl]-4-(3-trifluoromethylphenyl)piperazine.

15-18. (canceled)

19. The heteroarylthio compound of claim 1, wherein the compound is selected from the group consisting of:
4-{4-[3-(1-methyl-1H-imidazol-2-ylsulfanyl)propyl]piperazin-1-yl}furo[3,2-c]pyridine;
4-{4-[3-(1-phenyl-1H-tetrazol-5-ylsulfanyl)propyl]piperazin-1-yl}furo[3,2-c]pyridine; and
4-{4-[3-(5-pyridin-4-yl-[1,3,4]oxadiazol-2-sulfanyl)propyl]piperazin-1-yl}furo[3,2-c]pyridine.

20-21. (canceled)

22. A pharmaceutical composition comprising the heteroarylthio compound of claim 1 and one or more pharmaceutically acceptable excipients.

23. A method of treating a neurological condition, comprising the step of administering the heteroarylthio compound of claim 1 to a subject in need thereof.

24. (canceled)
